(19) United States
(12) Patent Application Publication
(10) Pub. No.: US 2005/0070972 A1
(43) Pub. Date: Mar. 31, 2005

(54) Energy shunt for producing an MRI-safe implantable medical device

(76) Inventors: Carl D. Wahlstrand, Lino Lakes, MN (US); Gregory A. Hrdlicka, Plymouth, MN (US); Robert M. Skime, Coon Rapids, MN (US); Piotr Przybyszewski, Coon Rapids, MN (US); Thomas E. Cross JR., St. Francis, MN (US)

Correspondence Address: INGRASSIA FISHER & LORENZ, P.C., 7150 E. CAMELBACK, STE. 325, SCOTTSDALE, AZ, 85251, US

(21) Appl. No.: 10/946,968

(22) Filed: Sep. 20, 2004

Related U.S. Application Data
(60) Provisional application No. 60/506,562, filed on Sep. 26, 2003.
(60) Provisional application No. 60/557,991, filed on Mar. 30, 2004.

(51) Int. Cl. A61N 1/18

(52) U.S. Cl. 607/48

(57) ABSTRACT

A neurostimulation system is configured for implantation into a patient's body and comprises a neurostimulator, a conductive stimulation lead having a first proximal end and a first distal end, at least one distal electrode electrically coupled proximate the first distal end, and a lead extension having a second proximal end electrically coupled to the neurostimulator and having a second distal end electrically coupled to the first proximal end. A shunt is electrically coupled to the first proximal end for diverting RF energy from the lead.

### CROSS-REFERENCES TO RELATED APPLICATIONS

This application claims the benefit of U.S. Provisional Application No. 60/506,562, filed Sep. 26, 2003 and U.S. Provisional Application No. 60/557,991, filed Mar. 30, 2004.

### FIELD OF THE INVENTION

The present invention generally relates to implantable medical devices, and more particularly to an energy shunt for use in conjunction with an implantable medical device such as a neurostimulation system which, when used in an MRI (Magnetic Resonance Imaging) environment, shunts energy at MRI frequencies to a patient's body in a safe manner.

### BACKGROUND OF THE INVENTION

Implantable medical devices are commonly used today to treat patients suffering from various ailments. Such implantable devices may be utilized to treat conditions such as pain, incontinence, sleep disorders, and movement disorders such as Parkinson's disease and epilepsy. Such therapies also appear promising in the treatment of a variety of physiological, emotional, and other psychological conditions.

One known type of implantable medical device, a neurostimulator, delivers mild electrical impulses to neural tissue using an electrical lead. For example, to treat pain, electrical impulses may be directed to specific sites. Such neurostimulation may result in effective pain relief and a reduction in the use of pain medications and/or repeat surgeries.

Typically, such devices are totally implantable and may be controlled by a physician or a patient through the use of an external programmer. Current systems generally include a non-rechargeable primary cell neurostimulator, a lead extension, and a stimulation lead, and the two main classes of systems may be referred to as: (1) Spinal Cord Stimulation (SCS) and (2) Deep Brain Stimulation (DBS).

An SCS stimulator may be implanted in the abdomen, upper buttock, or pectoral region of a patient and may include at least one extension running from the neurostimulator to the lead or leads which are placed somewhere along the spinal cord. Each of the leads (to be discussed in detail hereinbelow) currently contains from one to eight electrodes. Each extension (likewise to be discussed in detail below) is plugged into or connected to the neurostimulator at a proximal end thereof and is coupled to and interfaces with the lead or leads at a distal end of the extension or extensions.

The implanted neurostimulation system is configured to send mild electrical pulses to the spinal cord. These electrical pulses are delivered through the lead or leads to regions near the spinal cord or the nerve selected for stimulation. Each lead includes a small insulated wire coupled to an electrode at the distal end thereof through which the electrical stimulation is delivered. Typically, the lead also comprises a corresponding number of internal wires to provide separate electrical connection to each electrode such that each electrode may be selectively used to provide stimulation. Connection of the lead to an extension may be accomplished by means of a connector block including, for example, a series or combination of set screws, ball seals, etc. The leads are inserted into metal set screw blocks, and metal set screws are manipulated to press the contacts against the blocks to clamp them in place and provide electrical connection between the lead wires and the blocks. Such an arrangement is shown in U.S. Pat. No. 5,458,629 issued Oct. 17, 1995 and entitled “Implantable Lead Ring Electrode and Method of Making”.

A DBS system comprises similar components (i.e. a neurostimulator, at least one extension, and at least one stimulation lead) and may be utilized to provide a variety of different types of electrical stimulation to reduce the occurrence or effects of Parkinson's disease, epileptic seizures, or other undesirable neurological events. In this case, the neurostimulator may be implanted into the pectoral region of the patient. The extension or extensions may extend up through the patient's neck, and the leads/electrodes are implanted in the brain. The leads may interface with the extension just above the ear on both sides of the patient. The distal end of the lead may contain from four to eight electrodes and, as was the case previously, the proximal end of the lead may be connected to the distal end of the extension and may be held in place by set screws. The proximal portion of the extension plugs into the connector block of the neurostimulator.

Magnetic resonance imaging (MRI) is a relatively new and efficient technique that may be used in the diagnosis of many neurological disorders. It is an anatomical imaging tool which utilizes non-ionizing radiation (i.e. no x-rays or gamma rays) and provides a non-invasive method for the examination of internal structure and function. For example, MRI permits the study of the overall function of the heart in three dimensions significantly better than any other imaging method. Furthermore, imaging with tagging permits the non-invasive study of regional ventricular function.

MRI scanning is widely used in the diagnosis and injuries to the head. In fact, the MRI is now considered by many to be the preferred standard of care, and failure to prescribe MRI scanning can be considered questionable. Approximately sixteen million MRIs were performed in 1996, followed by approximately twenty million in the year 2000. It is projected that forty million MRIs will be performed in 2004.

In an MRI scanner, a magnet creates a strong magnetic field which aligns the protons of hydrogen atoms in the body and then exposes them to radio frequency (RF) energy from a transmitter portion of the scanner. This spins the various protons, and they produce a faint signal that is detected by a receiver portion of the scanner. A computer renders these signals into an image. During this process, three electromagnetic fields are produced; i.e. (1) a static magnetic field, (2) a gradient magnetic field, and (3) a radio frequency (RF) magnetic field. The main or static magnetic field may typically vary between 0.2 and 3.0 Tesla. A nominal value of 1.5 Tesla is approximately equal to 15,000 Gauss which is 30,000 times greater than the Earth's magnetic field of approximately 0.5 Gauss. The time varying or gradient magnetic field may have a maximum strength of approximately 40 milli-Tesla/meter at a frequency of 0-5 KHz. The RF may, for example, produce thousands of watts at frequencies of between 8-128 MHz. For example, up to 20,000 watts may be produced at 64 MHz and a static magnetic field of 1.5 Tesla; that is, 20 times more power than a typical toaster. Thus, questions have arisen regarding the potential risk associated with undesirable interaction between the MRI environment and the above-described neurostimulation systems; e.g. forces and torque on the implantable device within the MRI scanner caused by the static magnetic field, RF-induced heating, induced currents due to gradient magnetic fields, device damage, and image distortion. Of these interactions, the problems associated with induced RF currents in the leads are most deserving of attention since it has been found that the temperature in the leads can rise by as much as 25° Centigrade or higher in an MRI environment.

Accordingly, it would be desirable to provide an implantable medical device that may be safely operated in an MRI environment. It would be further desirable to provide an implantable medical device such as a SCS or DBS neurostimulation system that may be operated in an MRI environment without the generation of significant heat in the leads due to induced RF currents. It would be further desirable to provide a component or insert that may be used in conjunction with known implantable medical devices that shunts induced RF currents induced at MRI frequencies away from the lead electrodes. Furthermore, other desirable features and characteristics of the present invention will become apparent from the subsequent detailed description of the invention and the appended claims, taken in conjunction with the accompanying drawings and this background of the invention.

### BRIEF SUMMARY OF THE INVENTION

According to a broad aspect of the invention, there is provided a neurostimulation system configured for implantation into a patient's body. The system comprises a neurostimulator, a conductive stimulation lead having a first proximal end and a first distal end, at least one distal electrode electrically coupled proximate the first distal end, and a lead extension having a second proximal end electrically coupled to the neurostimulator and having a second distal end electrically coupled to the first proximal end. A shunt is electronically coupled to the first proximal end for diverting RF energy from the lead.

According to a still further aspect of the invention there is provided a method for diverting RF energy induced during an MRI scan in a lead assembly implanted in a patient's body, the lead assembly including a distal electrode. A shunt having a first end coupled to the lead is implanted and is configured to divert the induced RF energy away from the lead and distal electrode at MRI frequencies.

### BRIEF DESCRIPTION OF THE DRAWINGS

The present invention will hereinafter be described in conjunction with the following drawing figures, wherein like numerals denote like elements, and:

FIG. 1 illustrates a typical spinal cord stimulation system implanted in a patient;

FIG. 2 illustrates a typical deep brain stimulation system implanted in a patient;

FIG. 3 is an isometric view of the distal end of the lead shown in FIG. 2;

FIG. 4 is an isometric view of the distal end of the extension shown in FIG. 2;

FIG. 5 is an isometric view of an example of a connector screw block suitable for connecting the lead of FIG. 3 to the extension shown in FIG. 4;

FIG. 6 is a top view of the lead shown in FIG. 2;

FIGS. 7 and 8 are cross-sectional views taken along lines 7-7 and 8-8 respectively in FIG. 6;

FIG. 9 is a top view of an alternate lead configuration;

FIGS. 10 and 11 are longitudinal and radial cross-sectional views respectively of a helically wound lead of the type shown in FIG. 6;

FIGS. 12 and 13 are longitudinal and radial cross-sectional views of a cabled lead;

FIG. 14 is an exploded view of a neurostimulation system;

FIG. 15 is a cross-sectional view of the extension shown in FIG. 14 taken along line 15-15;

FIG. 16 is a functional diagram illustrating how MRI-induced RF energy may be shunted away from an implanted lead;

FIG. 17 is an isometric view of a first embodiment of the inventive shunt insert;

FIG. 18 illustrates an exemplary embodiment of the inventive shunt insert utilizing a capacitive array; and

FIG. 19 is a schematic diagram illustrating how impedance matching may be utilized to optimize the diversion of induced RF energy away from the lead.

### DETAILED DESCRIPTION OF THE INVENTION

The following detailed description of the invention is merely exemplary in nature and is not intended to limit the invention or the application and uses of the invention. Furthermore, there is no intention to be bound by any theory presented in the preceding background of the invention or the following detailed description of the invention.

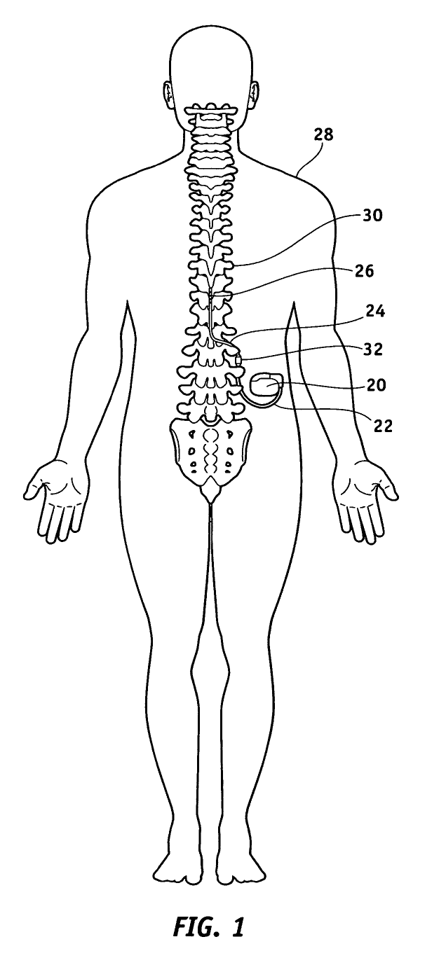

FIG. 1 illustrates a typical SCS system implanted in a patient. As can be seen, the system comprises a pulse generator such as an SCS neurostimulator 20, a lead extension 22 having a proximal end coupled to neurostimulator 20 as will be more fully described below, and a lead 24 having proximal end coupled to the distal end of extension 22 and having a distal end coupled to one or more electrodes 26. Neurostimulator 20 is typically placed in the abdomen of a patient 28, and lead 24 is placed somewhere along spinal cord 30. As stated previously, neurostimulator 20 may have one or two leads each having four to eight electrodes. Such a system may also include a physician programmer and a patient programmer (not shown). Neurostimulator 20 may be considered to be an implantable pulse generator of the type available from Medtronic, Inc. and capable of generating multiple pulses occurring either simultaneously or one pulse shifted in time with respect to the other, and having independently varying amplitudes and pulse widths. Neurostimulator 20 contains a power source and the electronics for sending precise, electrical pulses to the spinal cord to provide the desired treatment therapy. While neurostimulator 20 typically provides electrical stimulation by way of pulses, other forms of stimulation may be used as continuous electrical stimulation.

Lead 24 is a small medical wire having special insulation thereon and includes one or more insulated electrical conductors each coupled at their proximal end to a connector and to contacts/electrodes 26 at its distal end. Some leads are designed to be inserted into a patient percutaneously (e.g. the Model 3487A Pisces—Quad® lead available from Medtronic, Inc.), and some are designed to be surgically implanted (e.g. Model 3998 Specify® lead, also available form Medtronic, Inc.). Lead 24 may contain a paddle at its distant end for housing electrodes 26; e.g. a Medtronic paddle having model number 3587A. Alternatively, electrodes 26 may comprise one or more ring contacts at the distal end of lead 24 as will be more fully described below.

While lead 24 is shown as being implanted in position to stimulate a specific site in spinal cord 30, it could also be positioned along the peripheral nerve or adjacent neural tissue ganglia or may be positioned to stimulate muscle tissue. Furthermore, electrodes 26 may be epidural, intrathecal or placed into spinal cord 30 itself. Effective spinal cord stimulation may be achieved by any of these lead placements. While the lead connector at proximal end of lead 24 may be coupled directly to neurostimulator 20, the lead connector is typically coupled to lead extension 22 as is shown in FIG. 1. An example of a lead extension is Model 7495 available from Medtronic, Inc.

A physician's programmer (not shown) utilizes telemetry to communicate with the implanted neurostimulator 20 to enable the physician to program and manage a patient's therapy and troubleshoot the system. A typical physician's programmer is available from Medtronic, Inc. and bears Model No. 7432. Similarly, a patient's programmer (also not shown) also uses telemetry to communicate with neurostimulator 20 so as to enable the patient to manage some aspects of their own therapy as defined by the physician. An example of a patient programmer is Model 7434® 3 EZ Patient Programmer available from Medtronic, Inc.

Implantation of a neurostimulator typically begins with the implantation of at least one stimulation lead usually while the patient is under a local anesthetic. While there are many spinal cord lead designs utilized with a number of different implantation techniques, the largest distinction between leads revolves around how they are implanted. For example, surgical leads have been shown to be highly effective, but require a laminectomy for implantation. Percutaneous leads can be introduced through a needle, a much easier procedure. To simplify the following explanation, discussion will focus on percutaneous lead designs, although it will be understood by those skilled in the art that the inventive aspects are equally applicable to surgical leads. After the lead is implanted and positioned, the lead's distal end is typically anchored to minimize movement of the lead after implantation. The lead's proximal end is typically configured to connect to a lead extension 22. The proximal end of the lead extension is then connected to the neurostimulator 20.

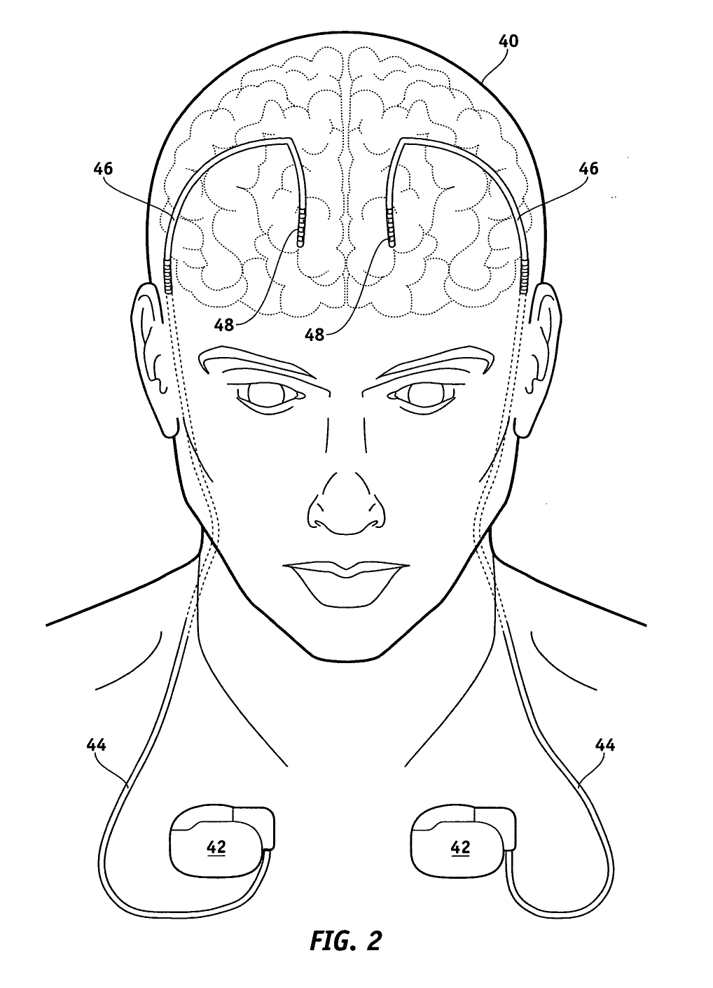

FIG. 2 illustrates a DBS system implanted in a patient 40 and comprises substantially the same components as does an SCS; that is, at least one neurostimulator, at least one extension, and at least one stimulation lead containing one or more electrodes. As can be seen, each neurostimulator 42 is implanted in the pectoral region of the patient. Extensions 44 are deployed up through the patient's neck, and leads 46 are implanted in the patient's brain is as shown at 48. As can be seen, each of the leads 46 is connected to its respective extension 44 just above the ear on both sides of patient 40.

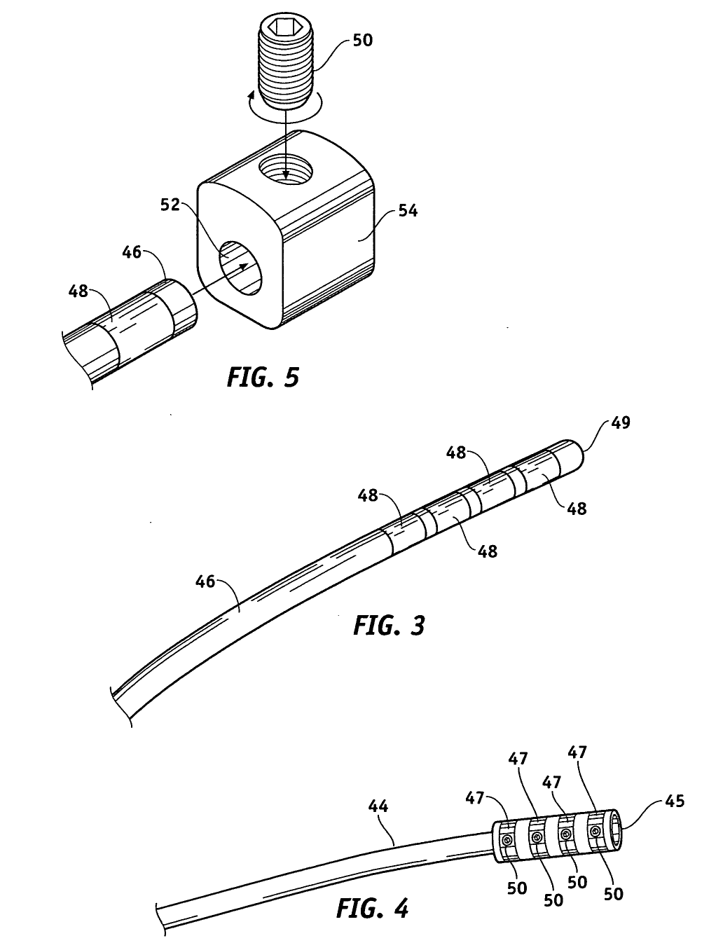

FIG. 3 is an isometric view of the distal end of lead 46. In this case, four ring electrodes 48 are positioned on the distal end of lead 46 and coupled to internal conductors of filers (not shown) contained within lead 46. Again, while four ring electrodes are shown in FIG. 3, it is to be understood that the number of electrodes can vary to suit a particular application. FIG. 4 is an isometric view of the distal end of extension 44, which includes a connector portion 45 having four internal contacts 47. The proximal end of the DBS lead is shown in FIG. 3, plugs into the distal connector 45 of extension 44, and is held in place by means of, for example, a plurality (e.g. four) of set screws 50. For example, referring to FIG. 5, lead 46 terminates in a series of proximal electrical ring contacts 48 (only one of which is shown in FIG. 5). Lead 46 may be inserted through an axially aligned series of openings 52 (again only one shown) in screw block 54. With a lead 46 so inserted, a series of set screws (only one shown) are screwed into block 54 to drive contacts 48 against blocks 54 and secure and electrically couple the lead 46. It should be appreciated, however, that other suitable methods for securing lead 46 to extension 44 may be employed. The proximal portion of extension 44 is secured to neurostimulator 42 as is shown in FIGS. 1 and 2.

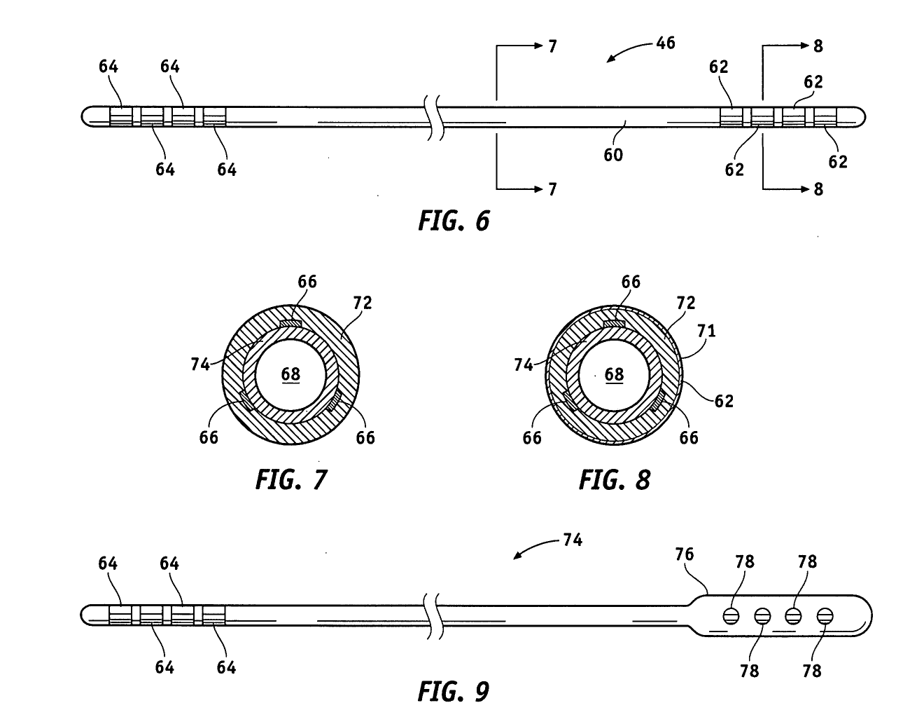

FIG. 6 is a top view of lead 46 shown in FIG. 2. FIGS. 7 and 8 are cross-sectional views taken along lines 7-7 and 8-8 respectively in FIG. 6. Distal end 60 of lead 46 includes at least one electrode 62 (four are shown). As stated previously, up to eight electrodes may be utilized. Each of electrodes 62 is preferably constructed as is shown in FIG. 8. That is, electrode 62 may comprise a conductive ring 71 on the outer surface of the elongate tubing making up distal shaft 60. Each electrode 62 is electrically coupled to a longitudinal wire 66 (shown in FIGS. 7 and 8) each of which extends to a contact 64 at the proximal end of lead 46. Longitudinal wires 66 may be of a variety of configurations; e.g. discreet wires, printed circuit conductors, etc. From the arrangement shown in FIG. 6, it should be clear that four conductors or filers run through the body of lead 46 to electrically connect the proximal electrodes 64 to the distal electrodes 62. As will be further discussed below, the longitudinal conductors 66 may be spirally configured along the axis of lead 46 until they reach the connector contacts.

The shaft of lead 46 preferably has a lumen 68 extending therethrough for receiving a stylet that adds a measure of rigidity during installation of the lead. The shaft preferably comprises a comparatively stiffer inner tubing member 70 (e.g. a polyamine, polyamide, high density polyethylene, polypropylene, polycarbonate or the like). Polyamide polymers are preferred. The shaft preferably includes a comparatively softer outer tubing member 72; e.g. silicon or other suitable elastomeric polymer. Conductive rings 71 are preferably of a biocompatible metal such as one selected from the noble group of metals, preferably palladium, platinum or gold and their alloys.

FIG. 9 illustrates an alternative lead 74 wherein distal end 76 is broader (e.g. paddle-shaped) to support a plurality of distal electrodes 78. A lead of this type is shown in FIG. 1. As was the case with the lead shown in FIGS. 6, 7, and 8, distal electrodes 78 are coupled to contacts 64 each respectively by means of an internal conductor or filer. A more detailed description of the leads shown in FIGS. 6 and 9 may be found in U.S. Pat. No. 6,529,774 issued Mar. 4, 2003 and entitled “Extradural Leads, Neurostimulator Assemblies, and Processes of Using Them for Somatosensory and Brain Stimulation”.

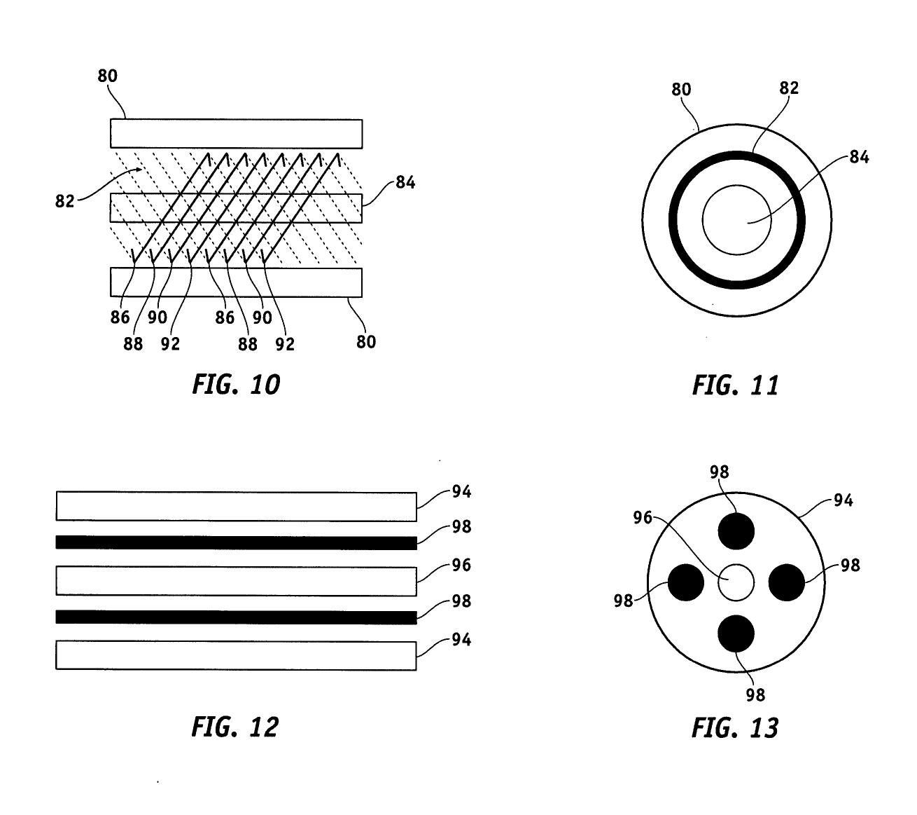

Leads of the type described above may be of the wound helix filer type or of the cabled filer type. FIGS. 10 and 11 are longitudinal and radial cross-sectional views respectively of a helically wound lead of the type shown in FIG. 6. The lead comprises an outer lead body 80; a plurality of helically wound, co-radial lead filers 82; and a stylet lumen 84. As stated previously, a stylet is a stiff, formable insert placed in the lead during implant so as to enable the physician to steer the lead to an appropriate location. FIG. 10 illustrates four separate, co-radially wound filers 86, 88, 90, and 92 which are electrically insulated from each other and electrically couple a single electrode 62 (FIG. 6) to a single contact 64 (FIG. 6).

As can be seen, the lead filers 82 have a specific pitch and form a helix of a specific diameter. The helix diameter is relevant in determining the inductance of the lead. These filers themselves also have a specific diameter and are made of a specific material. The filer diameter, material, pitch and helix diameter are relevant in determining the impedance of the lead. In the case of a helically wound lead, the inductance contributes to a frequency dependent impedance. FIGS. 12 and 13 are longitudinal and radially cross-sectional views respectively of a cabled lead. The lead comprises outer lead body 94, stylet lumen 96, and a plurality (e.g. four to eight) of straight lead filers 98.

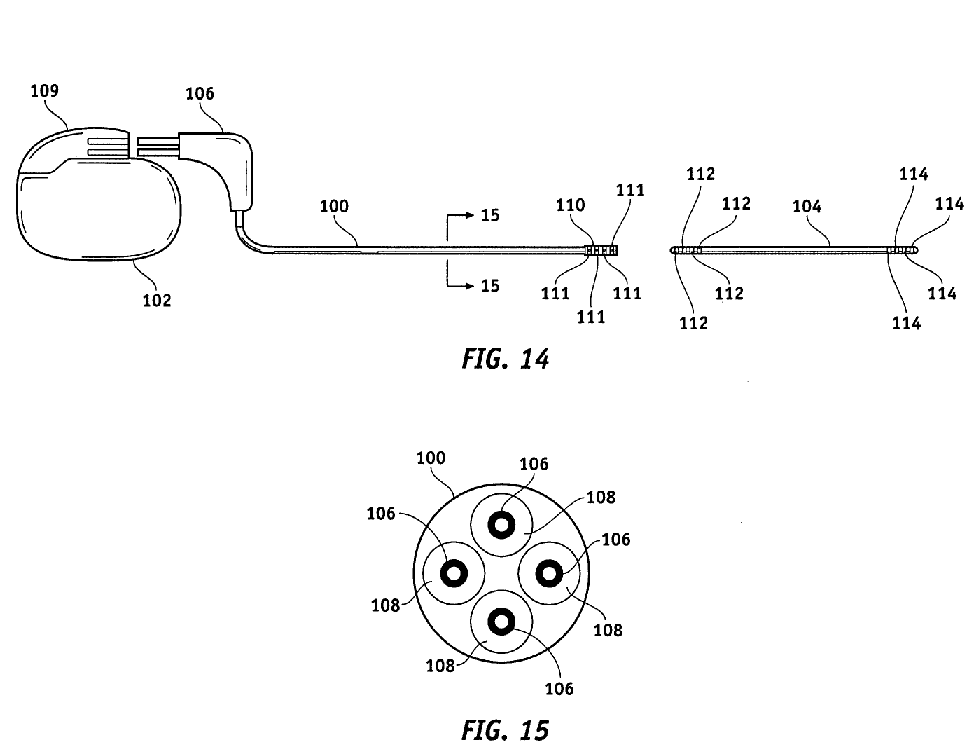

FIG. 14 is an exploded view of a neurostimulation system that includes an extension 100 configured to be coupled between a neurostimulator 102 and lead 104. The proximal portion of extension 100 comprises a connector 106 configured to be received or plugged into connector block 109 of neurostimulator 102. The distal end of extension 100 likewise comprises a connector 110 including internal contacts 111 and is configured to receive the proximal end of lead 104 having contacts 112 thereon. The distal end of lead 104 includes distal electrodes 114. FIG. 15 is a cross-sectional view of extension 100. Lead extension 100 has a typical diameter of 0.1 inch, which is significantly larger than that of lead 104 so as to make extension 100 more durable than lead 104. Extension 100 differs from lead 104 also in that each filer 106 in lead body is helically wound or coiled in its own lumen 108 and not co-radial with the rest of the filers as was the case in lead 104.

The diameter of typical percutaneous leads is approximately 0.05 inch. This diameter is based upon the diameter of the needle utilized in the surgical procedure to deploy the lead and upon other clinical anatomical requirements. The length of such percutaneous SCS leads is based upon other clinical anatomical requirements. The length of such percutaneous SCS leads is typically 28 centimeters; however, other lengths are utilized to meet particular needs of specific patients and to accommodate special implant locations.

Lead length is an important factor in determining the suitability of using the lead in an MRI environment. For example, the greater length of the lead, the larger the effective loop area that is impacted by the electromagnetic field (e.g. the longer the lead, the larger the antenna). Furthermore, depending on the lead length, there can be standing wave effects that create areas of high current along the lead body. This can be problematic if the areas of high current are near the distal electrodes.

Compared to the helically wound lead, the cable lead has smaller DC resistance because the length of the straight filer is less than that of a coiled filer and the impedance at high frequency is reduced because the inductance has been significantly reduced. It has been determined, that the newer cabled filer designs tend to be more problematic in an MRI environment than do the wound helix filer designs. It should be noted that straight filers for cable leads sometimes comprise braided stranded wire that includes a number of smaller strands woven to make up each filer. This being the case, the number of strands could be varied to alter the impedance.

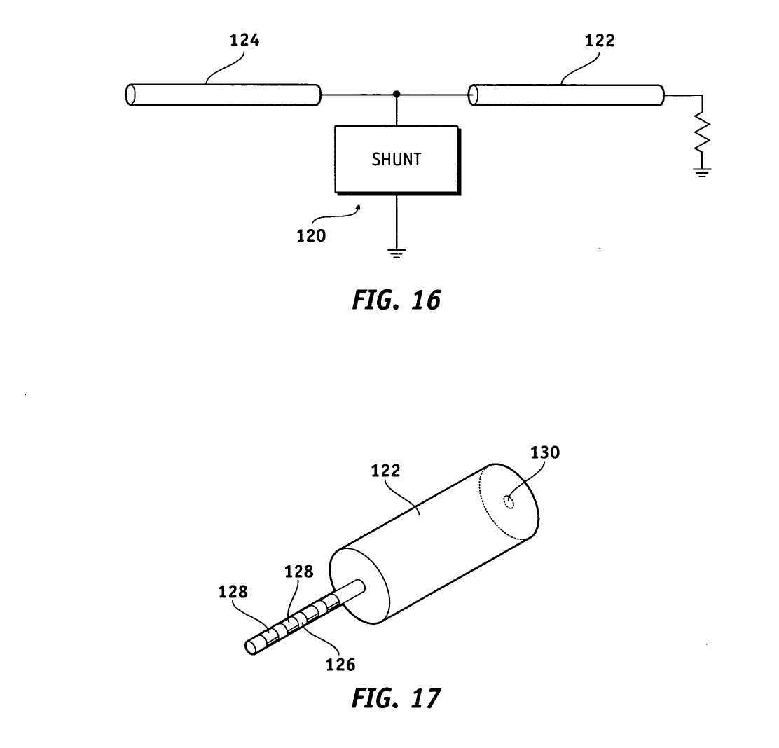

As stated previously, the electromagnetic fields within an MRI environment produce RF currents in the leads that can result in undesirable temperature increases at the lead electrodes. An arrangement for minimizing this problem is shown in FIG. 16. As can be seen, an insert or shunt shown generally at 120 is coupled to the junction of stimulation lead 122 and extension 124. Insert 120 functions to shunt energy induced in lead 122 during an MRI scan to the patient's body in a safe manner. This may be accomplished by shunting the energy to a large surface area electrode that is in contact with the body (i.e. an electrode having a large enough surface area to result in a low current density thus reducing heating). To accomplish this, the insert may utilize a combination of passive and/or active elements to safely divert the energy. Filters such as high-pass and band-pass filters are known and may readily be configured to accomplish the desired result. For example, a high-pass or capacitive filter may be used to shunt the energy to the large surface area (i.e. indifferent) electrode. Such a device is configured to appear as an open circuit at stimulation frequencies and as a short circuit to the indifferent electrode at MRI frequencies. The shunt may be designed to have an input impedance that matches the characteristic impedance of the lead thus (i) maximizing the energy transfer from the lead to the shunt and therefore to the indifferent electrode and (ii) reducing unwanted reflections. If desired, the shunt insert may utilize active electronics that may be powered from energy created in the lead itself during an MRI scan or from the implantable device itself.

Since the shunt insert is placed between the junction of lead 122 and extension 124, it should be clear that it has the capability of being utilized with existing leads, extensions, and implantable pulse generators. Thus, existing systems may be retrofitted with the shunt insert. This solution is also capable of being incorporated with abandoned leads; i.e. leads that have been left in a patient's body. For example, sometimes it is necessary to remove an implanted stimulation device and replace it with a newer device, but not necessarily at the same time. The lead may be left in the patient's body for some time prior to implanting the new device. By utilizing the inventive shunt insert in connection with the abandoned lead, the lead and its electrodes are rendered safe from unwanted heating during an MRI. In some cases, a lead is implanted and an MRI scan is desired prior to coupling the lead to a stimulation device. Again, the inventive shunt insert can be utilized in conjunction with the lead to render it MRI-safe. Finally, existing implanted neurostimulation systems may be retrofitted with the inventive shunt insert when, for example, it becomes necessary to replace the battery.

FIG. 17 is an isometric view of an exemplary embodiment of the inventive shunt insert. As can be seen, the insert comprises an extension interface 126 having a plurality of ring contacts 128 thereon, a lead interface 130 for receiving and making electrical contact with ring contacts on the proximal end of lead 122 (contacts 112 in FIG. 14), and an insert body 132, which may itself be made of a conductive material such as, for example, titanium. In this case, body 132 may act as the indifferent electrode. The distal electrode of a typical percutaneous lead is approximately twelve square millimeters. The indifferent electrode has a surface area at least an order of magnitude higher; i.e. approximately one hundred and twenty square millimeters.

While the example shown in FIG. 19 represents a basic matching network for a single frequency, other design solutions that match over a wide range of frequencies may be designed by the skilled practitioner. This may be thought of conceptually as replacing resistor 138 with a more complicated RLC network. This design may include active components that measure the characteristic impedance and adjust the matching resistance appropriately.

The use of a biocompatible hermetic conducting enclosure to protect the traces and components within the insert body has certain advantages. For example, due to its strength, it can be relatively thin (e.g. 0.01 inch thick). It may however require hermetic feedthroughs which will increase the size of the package. Alternatively, insert body 132 may be constructed of a non-conducting hermetic material such as ceramic that includes a large conducting portion (e.g. titanium) that serves as the indifferent electrode. The titanium contact could be, for example, brazed to the ceramic to form the indifferent electrode. The use of a ceramic body is advantageous in that vias can be constructed through the ceramic thus eliminating the need for feedthroughs. However, to achieve the desired strength, the ceramic package must be thicker than its titanium counterpart (e.g. 0.035 inch).

A ceramic piece may be created having the proper wire routes and bonding pads for a capacitive array. The array could then be placed on the ceramic piece and the assembly hermetically sealed by placing gold over the capacitive array. The gold piece could also act as the indifferent electrode in this configuration.

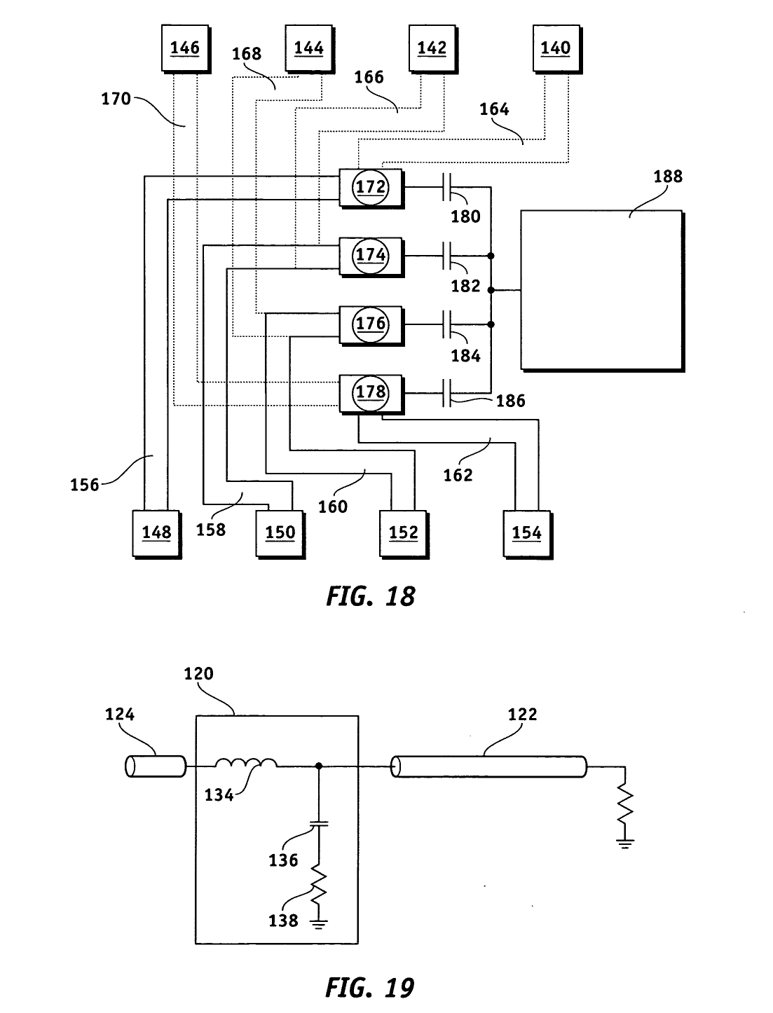

FIG. 18 is a plan view of a capacitive array suitable for encapsulation use in the inventive shunt insert. The circuit shown in the FIG. 18 includes lead contact sites 140, 142, 144, and 146 which are electrically coupled to extension contact sites 148, 150, 152, and 154. The design shown in FIG. 18 is two-sided thus minimizing the dimensions of the insert. Top traces 156, 158, 160, and 162 are shown with solid lines, and bottom traces 164, 166, 168, and 170 are shown with dotted lines. Traces 156, 158, 160, and 162 are electrically coupled to traces 164, 166, 168 and 170 respectively by means of vias 172, 174, 176, and 178. The inner connection of the extension contacts and the lead contacts is made easier since the lead contacts and the extension contacts are on opposite sides of the board.

The capacitor array comprises capacitors 180, 182, 184, and 186. First terminals of capacitors 180, 182, 184, and 186 are likewise coupled to vias 172, 174, 176, and 178, respectively. The second terminal of capacitors 180, 182, 184, and 186 are electrically coupled to floating electrode 188.

The maximum stimulation frequency is in the order of 1000 Hz which is roughly four orders of magnitude lower than an MRI frequency of 43 MHz. This provides a great deal of flexibility in the design of a high-pass filter. A 1000 pF capacitor creates a high-pass filter that performs as an open circuit for direct current and stimulation frequencies and performs as a short circuit at MRI frequencies. However, capacitors in the range of 200 pF to 47,000 pF are also suitable, the goal being that the impedance at low frequencies should be significantly higher than the impedance along the path of the lead and significantly lower at high frequencies than the impedance along the path of the lead.

While the design shown in FIG. 18 is intended to be used in conjunction with leads having four conductors, the design may be modified to accommodate eight conductors by simply adding the additional four connections. Finally, a four capacitor, 1000 pF array suitable for use in the design shown in FIG. 18 is available from AVX Corp. located in Myrtle Beach, S.C.

FIG. 19 is useful in describing how impedance matching may be utilized to minimize induced energy reflections back towards the lead and therefore the lead electrodes. Referring to FIG. 19, shunt 120 includes inductor 134, a capacitor 136, and a resistor 138. The characteristic impedance of lead 122 may be calculated using Equation (1)

$$Z = \sqrt{\frac{R + j ω L}{G + j ω C}} \qquad Equation ( 1 )$$

wherein Z represents the characteristic impedance of the lead, R is the resistance along the filer or conductor, L is the inductance along the filer or conductor, C is the capacitance between the filer and the insert body, and G is the conductance between the filer and the insert body. The shunt circuit can be designed so that it has operational significance only during high frequency events. That is, during low frequency excitation, inductor 134 appears as a short circuit and capacitor 136 as an open circuit. Thus, resistor 138 has no significance. During high frequency excitation, however, inductor 134 acts as an open circuit and capacitor 36 as a short circuit. This places resistor 138 in the circuit and if it has the same impendence as the characteristic impedance of lead 122, energy reflected back toward lead 122 is minimized.

The inventive shunt insert is capable of interfacing with current neurostimulation systems. That is, extension interface 126 plugs into currently known extensions and receptor 130 is provided for receiving the proximal contacts of known stimulation leads. Lead acceptor 130 may be provided with a ball-seal and single set screw or, if desired, four such ball-seal connections. Extension interface 126 should mimic the proximal end of the lead that, but for the shunt insert, would be plugged into the extension.

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention, it being understood that various changes may be made in the function and arrangement of elements described in an exemplary embodiment without departing from the scope of the invention as set forth in the appended claims.

1. A neurostimulation system configured for implantation into a patient's body, the system comprising:
a neurostimulator;
a conductive stimulation lead having a first proximal end and a first distal end;
at least one distal electrode electrically coupled proximate said first distal end;
a lead extension having a second proximal end electrically coupled to said neurostimulator and having a second distal end electrically coupled to said first proximal end; and
a shunt electrically coupled to said first proximal end and for diverting RF energy from said lead.

2. A neurostimulation system according to claim 1 wherein the RF energy is induced during an MRI scan.

3. A neurostimulation system according to claim 1 wherein the RF energy is diverted at high frequency.

4. A neurostimulation system according to claim 3 wherein the high frequency is in the range of approximately 43 MHz to 128 MHz.

5. A neurostimulation system according to claim 1 wherein said shunt comprises a first contact configured for contacting the patient's body tissue.

6. A neurostimulation system according to claim 5 wherein said distal electrode has a first contact area and said first contact has a second surface area at least an order of magnitude greater than the first contact area.

7. A neurostimulation system according to claim 1 wherein said shunt comprises:
a high-pass filter having an input coupled to said first proximal end; and
a first contact coupled to an output of said high-pass filter and having a surface area greater than that of said distal electrode.

8. A neurostimulation system according to claim 7 wherein said lead comprises at least one conductor and wherein said filter comprises at least one capacitor.

9. A neurostimulation system according to claim 8 wherein said filter comprises a plurality of capacitors.

10. A neurostimulation system according to claim 9 wherein said lead comprises at least four conductors and wherein said filter comprises at least four capacitors each coupled to one of said at least four conductors.

11. A neurostimulation system according to claim 8 wherein said capacitor has a capacitance in the range of 200 pF to 47,000 pF.

12. A neurostimulation system according to claim 11 wherein said capacitor has a capacitance of approximately 1000 pF.

13. A neurostimulation system according to claim 5 wherein said shunt is hermetically sealed in a titanium body.

14. A neurostimulation system according to claim 5 wherein said shunt is hermetically sealed in a ceramic body.

15. A neurostimulation system according to claim 5 wherein said shunt is potted.

16. A neurostimulation system according to claim 13 wherein said titanium body comprises said first contact.

17. A neurostimulation system according to claim 14 wherein said first contact is on said ceramic body.

18. A neurostimulation system according to claim 1 wherein said shunt has a characteristic impedance substantially equal to the characteristic impedance of said lead.

19. An implantable lead assembly of the type utilized in conjunction with an implantable pulse generator, said lead assembly comprising:
a conductive stimulation lead having a first proximal end and a first distal end;
at least one distal electrode electrically coupled proximate said first distal end; and
a shunt electrically coupled to said first proximal for diverting RF energy from said lead.

20. An implantable lead assembly according to claim 19 wherein the RF energy in induced during an MRI scan at MRI frequencies.

21. An implantable lead assembly according to claim 20 wherein MRI frequencies are in the range of approximately 43 MHz to 128 MHz.

22. An implantable lead assembly according to claim 19 wherein said shunt comprises a first contact configured for contacting the patient's body tissue.

23. An implantable lead assembly according to claim 22 wherein said distal electrode has a first contact area and said first contact has a second surface area at least an order of magnitude greater than the first contact area.

24. An implantable lead assembly according to claim 19 wherein said shunt comprises:
a high-pass filter having an input coupled to said first proximal end; and
a first contact coupled to an output of said high-pass filter and having a surface area substantially greater than that of said distal electrode.

25. An implantable lead assembly according to claim 24 wherein said lead comprises at least one conductor and wherein said filter comprises at least one capacitor.

26. An implantable lead assembly according to claim 25 wherein said filter comprises a plurality of capacitors.

27. An implantable lead assembly according to claim 25 wherein said capacitor has a capacitance in the range of 200 pF to 47,000 pF.

28. An implantable lead assembly according to claim 27 wherein said capacitor has a capacitance of approximately 1000 pF.

29. An implantable lead assembly according to claim 21 wherein said shunt is hermetically sealed in a titanium body.

30. An implantable lead assembly according to claim 21 wherein said shunt is hermetically sealed in a ceramic body.

31. An implantable lead assembly according to claim 29 wherein said titanium body comprises said first contact.

32. An implantable lead assembly according to claim 19 wherein said shunt has a characteristic impedance substantially equal to the characteristic impedance of said lead.

33. A method for diverting RF energy induced, during an MRI scan, in a lead assembly implanted in a patient's body, the lead assembly including a distal electrode, the method comprising:
implanting a shunt having a first end coupled to the lead and configured to divert the induced RF energy from the lead and distal electrode at MRI frequencies.

34. A method according to claim 33 further comprising diverting the induced RF energy to the patient's body tissue.

35. A pulse stimulation system configured for implantation into a patient's body, the system comprising:
a pulse generator;
a conductive stimulation lead having a proximal end electrically coupled to said pulse generator and having a distal end;
at least one distal electrode electrically coupled proximate said distal end; and
a shunt electrically coupled to said proximal end for diverting RF energy from said lead.

36. A pulse stimulation system according to claim 35 wherein the RF energy is induced during an MRI scan.

37. A pulse stimulation system according to claim 35 wherein said shunt comprises a first contact configured for contacting the patient's body tissue.

38. A pulse stimulation system according to claim 37 wherein said distal electrode has a first surface area and said first contact has a second surface area at least an order of magnitude greater than the first surface area.

39. A pulse stimulation system according to claim 35 wherein said shunt comprises:
a high-pass filter having an input coupled to said proximal end; and
a first contact coupled to an output of said high-pass filter and having a surface area greater than that of said distal electrode.

40. A pulse stimulation system according to claim 39 wherein said lead comprises at least one conductor and wherein said filter comprises at least one capacitor.

41. A pulse stimulation system according to claim 40 wherein said capacitor has a capacitance in the range of 200 pF to 47,000 pF.

42. A pulse stimulation system according to claim 41 wherein said capacitor has a capacitance of approximately 1000 pF.

43. A pulse stimulation system according to claim 37 wherein said shunt is hermetically sealed in a titanium body.

44. A pulse stimulation system according to claim 43 wherein said titanium body comprises said first contact.

45. A pulse stimulation system according to claim 35 wherein said shunt has a characteristic impedance substantially equal to the characteristic impedance of said lead.
